(19) United States
(12) Patent Application Publication
(10) Pub. No.: US 2015/0309020 A1
(43) Pub. Date: Oct. 29, 2015

(54) SURFACE AND METHOD FOR SENSING PROTEIN-LIGAND BINDING

(71) Applicant: The Texas A&M University System, College Station, TX (US)

(72) Inventors: Paul S. Cremer, State College, PA (US); Aaron D. Robison, Abilene, TX (US); Da Huang, Houston, TX (US)

(73) Assignee: THE TEXAS A&M UNIVERSITY SYSTEM, College Station, TX (US)

(21) Appl. No.: 14/599,261

(22) Filed: Jan. 16, 2015

Related U.S. Application Data
(60) Provisional application No. 61/928,443, filed on Jan. 17, 2014.

(51) Int. Cl. G01N 33/543

(52) U.S. Cl. CPC G01N 33/54393

(57) ABSTRACT

Method for sensing the binding of an analyte to a surface using a pH-sensitive fluorescent agent having a first fluorescence emission intensity at a first pH and a second fluorescence emission intensity at a second pH, and surfaces for sensing the binding of an analyte to a surface using the pH-sensitive fluorescent agent.

### CROSS-REFERENCE TO RELATED APPLICATION

This application claims the benefit of U.S. Provisional Application No. 61/928,443, filed Jan. 17, 2014, which application is incorporated herein by reference in its entirety.

### STATEMENT OF GOVERNMENT LICENSE RIGHTS

This invention was made with Government support under Grant No. N00014-08-1-0467 awarded by the Office of Naval Research. The Government has certain rights in the invention.

### BACKGROUND OF THE INVENTION

The ability to monitor ligand-receptor interactions is vital for biotechnological advances as well as for a fundamental understanding of cell biology. The single-molecule sensitivity that fluorescent labeling can provide and the relative ease of fluorescence measurements has resulted in the utilization of this technique for detecting and monitoring proteins and nucleotides. Dye labels can, however, interfere with analyte activity and be difficult to efficiently employ. Many sensing techniques have consequently been developed such as quartz-crystal microbalance (QCM), surface plasmon resonance (SPR), semiconductor nanowire conductivity, and optical microcavities that avoid the use of fluorescent labels. Although these techniques avoid the problems associated with directly attaching fluorescent tags to analytes, they can behave non-linearly in response to analyte concentration, require specialized equipment or procedures which can be difficult and/or costly to employ, or possess poorer detection limits than fluorescence-based techniques.

It is advantageous to be able to monitor ligand-receptor interactions on supported lipid bilayers because these platforms effectively act as simplified cellular surfaces, incorporating the same lipid molecules as well as maintaining two-dimensional lipid fluidity. Indeed, the majority of current drug targets are receptor sites associated with the cell surface. This makes the ability to effectively monitor such systems key for therapeutics and drug discovery. Consequently, much work has been done to produce supported lipid bilayers that mimic the cell surface with various supports and tethers. By embedding these bilayers into microfluidic devices, one can probe binding ligands rapidly while utilizing only small amounts of analyte. Heterogeneous flow-based microfluidic techniques also enable the user to easily modulate parameters such as ionic strength and buffer pH while monitoring the fluorescent response in real time. The ability to utilize glass substrates as membrane supports is also advantageous as these surfaces can readily accommodate fluid lipid bilayers via vesicle fusion.

Recently, a sensing technique has been developed that employs the sensitivity of fluorescent measurements while avoiding the complications incurred by directly labeling the analyte of interest (Jung H, Robison A D, Cremer P S (2009) Detecting Protein-Ligand Binding on Supported Bilayers by Local pH Modulation. J Am Chem Soc 131 (3):1006-1014. This technique involves the incorporation of a pH sensitive dye, ortho-conjugated TEXAS RED® DHPE (TEXAS RED® 1,2-dihexadecanoyl-sn-glycero-3-phosphoethanolamine, triethylammonium salt), into a supported lipid bilayer. This isomer of TEXAS RED® loses fluoresces when it is deprotonated at higher pH. On the other hand, the para-conjugated TEXAS RED® isomer retains its fluorescence under basic conditions and therefore can serves as a reference dye (Corrie J E T, Davis C T, Eccleston J F (2001) Chemistry of sulforhodamine-amine conjugates. Bioconjugate Chem 12 (2):186-194). These TEXAS RED®-containing bilayers can also accommodate binding ligands such as biotin-PE (1,2-dipalmitoyl-sn-glycero-3-phosphoethanolamine-N-(cap biotinyl) (sodium salt)). Upon the binding of a negatively charged protein, the surface potential near the binding site will be decreased. This helps partition hydronium ions to the interface, which in turn causes protonation of the ortho-TEXAS RED® isomer and increases the fluorescence response. This increase sensing approach was demonstrated using the biotin/anti-biotin binding pair, with pM sensitivity shown.

Despite the advancement of sensing methods noted above, a need exists for developing sensing methods having increased sensitivity and utility. The present invention seeks to fulfill this need and provides further related advantages.

### SUMMARY OF THE INVENTION

In one aspect, the invention provides a method for sensing the binding of an analyte to a surface. In one embodiment, the method includes:

(a) contacting an analyte with a surface, wherein the analyte is a second binding partner of a binding pair, wherein the surface comprises a first binding partner of the binding pair having a binding affinity toward the second binding partner, and wherein the surface further comprises a pH-sensitive fluorescent agent having a first fluorescence emission intensity at a first pH and a second fluorescence emission intensity at a second pH; and

(b) measuring the fluorescence emission intensity of the pH-sensitive fluorescent agent after contacting the surface with the analyte, wherein a change in the fluorescent emission intensity after contacting the surface with the analyte indicates binding of the analyte to the surface.

In one embodiment, the surface is a lipid bilayer. The lipid bilayer can be a component of a liposome and the sensing of the binding of the analyte to the surface occurs in bulk solution. In another embodiment, the surface is a supported lipid bilayer. In this embodiment, the lipid bilayer can be formed on a variety of surfaces, including surfaces of fluidic devices such as microfluidic devices. Suitable surfaces include sensor surfaces.

Representative pH-sensitive fluorescent agents useful in the method of the invention are advantageously operative over a pH range from about pH 5.5 to about pH 9.0. In certain embodiments, the pH-sensitive fluorescent agent is covalently coupled to the lipid bilayer. In other embodiments, the pH-sensitive fluorescent agent is covalently coupled to a component making up the lipid bilayer. The pH-sensitive fluorescent agents useful in the invention have emission intensities that differ as a function of pH (e.g., first and second emission intensities that are not the same). In certain embodiments, the first fluorescence emission intensity is greater than the second fluorescence emission intensity. In other embodiments, the first fluorescence emission intensity is less than the second fluorescence emission intensity. Representative pH-sensitive fluorescent agents include rhodamine agents. In certain embodiments, the pH-sensitive fluorescent agent is a rhodamine B. In one embodiment, the rhodamine B is an ortho-rhodamine B.

In certain embodiments, the first binding partner is a ligand and the second binding partner is a receptor (e.g., a protein).

In another aspect of the invention, a sensor surface is provided. In one embodiment, the invention provides a sensor surface comprising:

(a) one or more first binding partners, each first binding partner having a binding affinity to a second binding partner of a binding pair; and

(b) one or more pH-sensitive fluorescent agents, wherein each pH-sensitive fluorescent agent has a first fluorescent emission intensity at a first pH and a second fluorescent emission intensity at a second pH, wherein the first and second emission intensities are not the same.

In a further aspect, the invention provides a method for detecting the presence of an analyte in a sample. In one embodiment, the method includes:

(a) contacting a sample with a surface, wherein the surface comprises a first binding partner of a binding pair having a binding affinity toward a second binding partner, wherein the analyte is the second binding partner, and wherein the surface further comprises a pH-sensitive fluorescent agent having a first fluorescence emission intensity at a first pH and a second fluorescence emission intensity at a second pH; and

(b) measuring the fluorescence emission intensity of the pH-sensitive fluorescent agent after contacting the surface with the sample, wherein a change in the fluorescent emission intensity after contacting the surface with the sample indicates binding of the analyte to the surface and the presence of the analyte in the sample.

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIG. 1A is a schematic illustration of pH modulation assays of the invention in both the decrease and increase mode; (a) illustrates a positively charged protein (e.g., avidin) recruiting hydroxide ions upon binding and rendering the membrane surface more basic, decreasing the fluorescence response of the dye; (b) illustrates a negatively charged protein (e.g., streptavidin) recruiting hydronium ions upon surface binding and enhancing the fluorescence response of nearby dye molecules.

FIG. 1B is a schematic illustration of a representative surface of the invention.

FIGS. 2A-2C illustrate the chemical structures of POPC, ortho-conjugated rhodamine B-POPE, and biotin-PE, respectively.

FIGS. 3A and 3B illustrate pH titration. FIG. 3A illustrates fluorescence intensity line scans across channels containing bilayers composed of 0.5 mol % ortho-rhodamine B POPE (oRB) in POPC with and without 5 mol % biotin-PE. FIG. 3B illustrates titration curves for each membrane composition. The y-axis values were obtained by dividing the intensity of the fluorescence line scan at each pH value by the maximum fluorescence intensity line scan at pH 4.5. The solid circles represent individual fluorescence measurements and the solid lines are sigmoidal fits to the data.

FIGS. 4A-4E illustrate protein sensing. FIG. 4A is a fluorescence image of a three channel bilayer-coated microfluidic device. The left and center channels contained supported bilayers containing 0.5 mol % ortho-rhodamine B and 5 mol % biotin PE in POPC. The right channel was nearly identical, but the bilayer lacked biotin PE. The dashed line corresponds to the region from which fluorescent intensity profile measurements were made before and after introduction of proteins. FIG. 4B compares average channel intensity as a function of time before and after the introduction of streptavidin (diamonds), avidin in the bilayer with biotin-PE (squares) and avidin in the bilayer without biotin-PE (triangles). Experimental line profiles of the microfluidic channels before and after the addition of 300 nM streptavidin (FIG. 4C), 300 nM avidin (FIG. 4D) and 300 nM avidin (FIG. 4E) into the right, center, and left channels, respectively. A 10× air objective was used for all measurements and experiments were run in 10 mM PBS buffer with 150 mM NaCl at a bulk pH of 7.2.

FIG. 5 compares titration curves for two buffer conditions with supported POPC bilayers containing 0.5 mol % ortho-rhodamine B and 5 mol % biotin-PE. The y-axis values were obtained from the intensity profiles at each pH divided by the maximum fluorescence intensity for the titration at low pH. The solid circles represent individual fluorescence measurements and the solid lines are sigmoidal fits to the data.

FIGS. 6A and 6B illustrate protein sensing in 10 mM PBS. Line scans for microfluidic channels before and after the introduction of streptavidin (FIG. 6A) and avidin (FIG. 6B). Referring to FIG. 6A a supported bilayer composed of 0.5 mol % ortho-rhodamine B and 5 mol % biotin-PE in POPC before and after the addition of streptavidin (respectively) (a) and a control bilayer composed of 0.5 mol % ortho-rhodamine B in POPC before and after addition of streptavidin (b). Referring to FIG. 6B a bilayer composed of 0.5 mol % ortho-rhodamine B and 5 mol % biotin-PE in POPC before and after the addition of avidin (c) and a control bilayer composed of 0.5 mol % ortho-rhodamine B in POPC before and after the addition of avidin (d).

FIG. 7 compares fluorescence intensity before and after the addition of streptavidin to buffers at pH 7.3 and 5.9. Both buffers contained 10 mM PBS and 150 mM NaCl. Each point represents the average fluorescence intensity inside the microfluidic channels as a function of time.

FIG. 8 is a matrix-assisted laser desorption ionization mass spectrum of rhodamine-B POPE+2H⁺ showing a peak at 1258.6.

FIG. 9 compares fluorescence intensity line profile data corresponding to a bilayer composed of 0.5 mol % ortho-rhodamine B-POPE in POPC before and after the addition of 300 nM streptavidin. The assay was run with 10 mM PBS with 150 mM NaCl at pH 7.2.

FIG. 10 compares fluorescence intensity line profile data corresponding to a bilayer composed of 5 mol % biotin and 0.5 mol % para-rhodamine B-POPE in POPC before and after the addition of 300 nM avidin. The assay was run with 10 mM PBS with 150 mM NaCl at pH 7.2.

FIG. 11 compares fluorescence intensity line profile data from which titration curves were extracted in FIG. 5.

FIG. 12 compares representative titration curves for various buffers and bilayer chemistries. NaOH and HCl were used to modulate the pH with each buffer used.

FIG. 13 compares fluorescence response relative to a normalized starting intensity of 1.00 for the addition of 300 nM streptavidin at pH 8.1 in buffers of varying ionic strength (5 mM, 10 mM, and 20 mM PBS). The assays were run with POPC bilayers containing 0.5 mol % ortho-rhodamine B-POPE and 5 mol % biotin-PE. The error bars correspond to the standard deviation of three fluorescent measurements.

### DETAILED DESCRIPTION OF THE INVENTION

In one aspect, the invention provides a method for sensing the binding of an analyte to a surface. In one embodiment, the method includes:

(a) contacting an analyte with a surface, wherein the analyte is a second binding partner of a binding pair, wherein the surface comprises a first binding partner of the binding pair having a binding affinity toward the second binding partner, and wherein the surface further comprises a pH-sensitive fluorescent agent having a first fluorescence emission intensity at a first pH and a second fluorescence emission intensity at a second pH; and

(b) measuring the fluorescence emission intensity of the pH-sensitive fluorescent agent after contacting the surface with the analyte, wherein a change in the fluorescent emission intensity after contacting the surface with the analyte indicates binding of the analyte to the surface.

The surface can be any one of a variety of surfaces suitable for use in the method. In one embodiment, the surface is a lipid bilayer. The lipid bilayer can be a component of a liposome and the sensing of the binding of the analyte to the surface occurs in bulk solution. In another embodiment, the surface is a supported lipid bilayer. In this embodiment, the lipid bilayer can be formed on a variety of surfaces, including surfaces of fluidic devices such as microfluidic devices. The surface can be the surface of a sensor.

Representative pH-sensitive fluorescent agents useful in the method of the invention are advantageously operative over a pH range from about pH 5.5 to about pH 9.0. The operable range of these fluorescent agents renders these fluorescent agents effective for a wide variety of assays. In certain embodiments, the pH-sensitive fluorescent agent is covalently coupled to the lipid bilayer. In other embodiments, the pH-sensitive fluorescent agent is covalently coupled to a component making up the lipid bilayer. The pH-sensitive fluorescent agents useful in the invention had emission intensities that differ as a function of pH (e.g., first and second emission intensities that are not the same). In certain embodiments, the first fluorescence emission intensity is greater than the second fluorescence emission intensity. In other embodiments, the first fluorescence emission intensity is less than the second fluorescence emission intensity.

Suitable pH-sensitive fluorescent agents include rhodamine agents. In certain embodiments, the pH-sensitive fluorescent agent is a rhodamine B. Representative rhodamine B agents include ortho-rhodamine B agents.

In one embodiment, the first binding partner is covalently coupled to the lipid bilayer. In one embodiment, the first binding partner is covalently coupled to a component making up the lipid bilayer (e.g., a lipid component). In this embodiment, the invention provides a conjugate of a lipid component and a pH-sensitive fluorescent agent. In certain embodiments, the first binding partner is a ligand and the second binding partner is a receptor (e.g., a protein). In other embodiments, the first binding partner is a receptor and the second binding partner is a ligand. In certain embodiments, the first binding partner is an antigen and the second binding partner is an antibody or fragment thereof. In other embodiments, the first binding partner is an antibody or fragment thereof and the second binding partner is an antigen. In certain embodiments, the first binding partner is a nucleic acid and the second binding partner is a nucleic acid.

In another aspect of the invention, a sensor surface is provided. In one embodiment, the invention provides a surface comprising:

(a) one or more first binding partners, each first binding partner having a binding affinity to a second binding partner of a binding pair; and

(b) one or more pH-sensitive fluorescent agents, wherein each pH-sensitive fluorescent agent has a first fluorescent emission intensity at a first pH and a second fluorescent emission intensity at a second pH, wherein the first and second emission intensities are not the same.

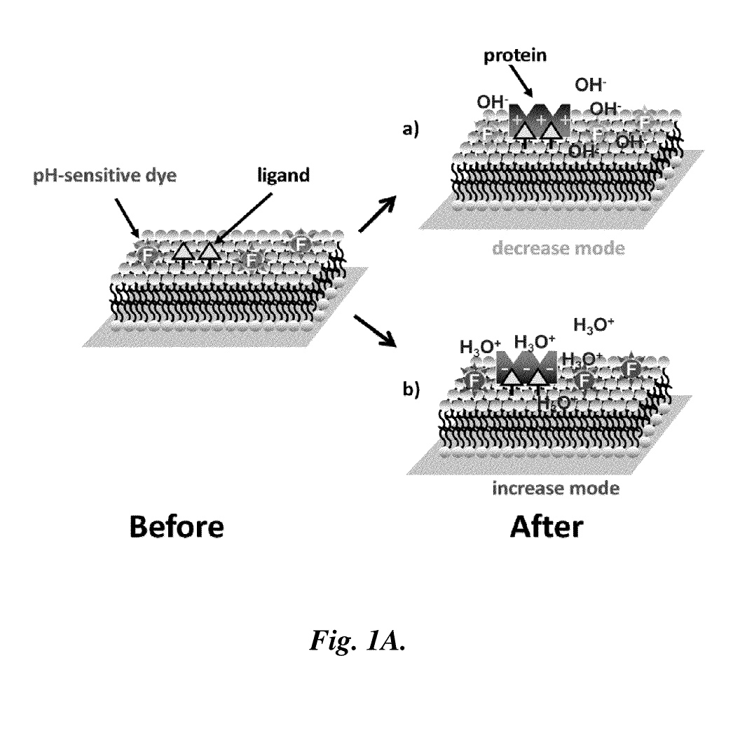

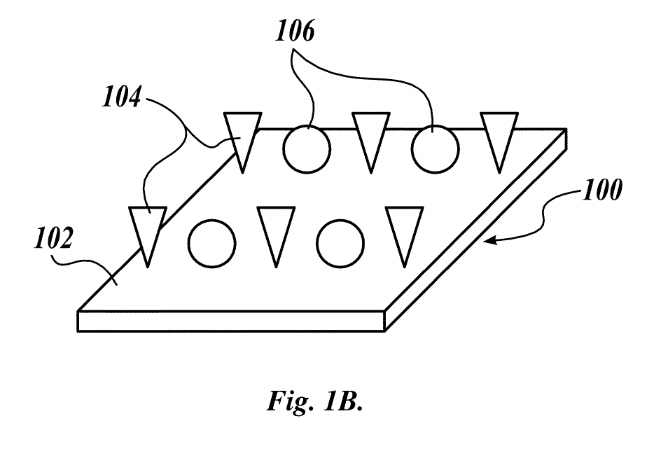

A representative surface of the invention 100 is illustrated in FIG. 1B. Referring to FIG. 1B, surface 102 includes a plurality of first binding partners 104 and a plurality of pH sensitive fluorescent agents 106. The surface can be a bilayer (see, for example, FIG. 1A).

In a further aspect, the invention provides a method for detecting the presence of an analyte in a sample. In one embodiment, the method includes:

(a) contacting a sample with a surface, wherein the surface comprises a first binding partner of a binding pair having a binding affinity toward a second binding partner, wherein the analyte is the second binding partner, and wherein the surface further comprises a pH-sensitive fluorescent agent having a first fluorescence emission intensity at a first pH and a second fluorescence emission intensity at a second pH; and

(b) measuring the fluorescence emission intensity of the pH-sensitive fluorescent agent after contacting the surface with the sample, wherein a change in the fluorescent emission intensity after contacting the surface with the sample indicates binding of the analyte to the surface and the presence of the analyte in the sample.

The following is a description of representative methods and surfaces of the invention.

pH Titration Curves

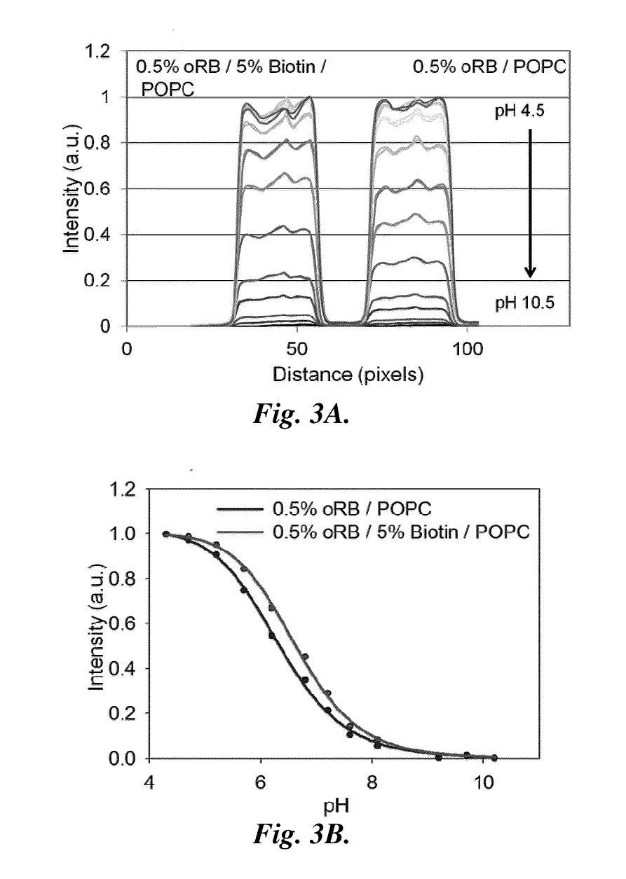

Initial experiments were performed to obtain titration curves for 0.5 mol % ortho-rhodamine B-POPE in POPC bilayers with 0 and 5 mol % biotin PE (FIGS. 3A and 3B). The pH of the bulk solution above the bilayers was altered via the continuous flow of pH adjusted 10 mM PBS solutions with 150 mM NaCl. The fluorescence was monitored until a steady state was reached. Once the fluorescent intensity stabilized at a given pH, the value was recorded and a different pH solution was flowed over the bilayer. Representative intensity line scans after reaching steady state can be found in FIG. 3A. The bilayer fluorescence data were normalized to the pH value with the maximum fluorescence intensity. Fluorescence intensity measurements versus pH value curves for the two bilayer conditions were fit to a sigmoidal curve (solid lines in FIG. 3B) and the apparent pKa values were 6.2±0.1 and 6.5±0.1, respectively, for bilayers with 0 mol % and 5 mol % biotin-PE.

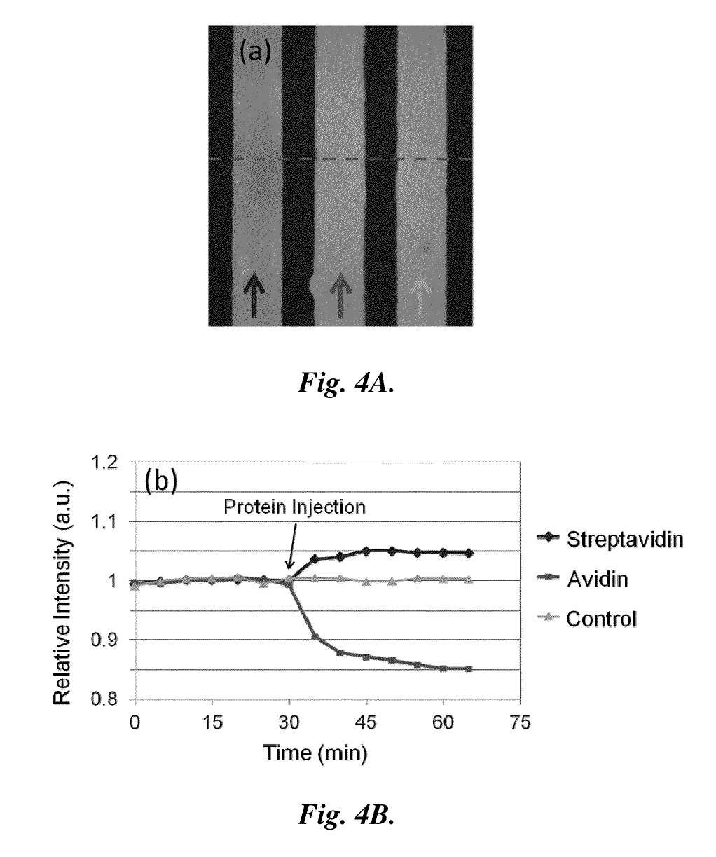

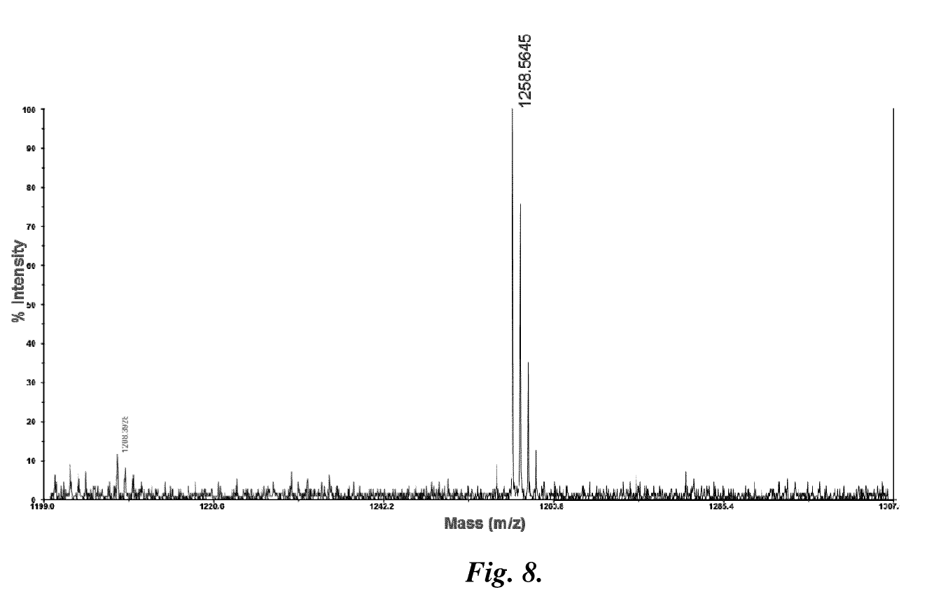

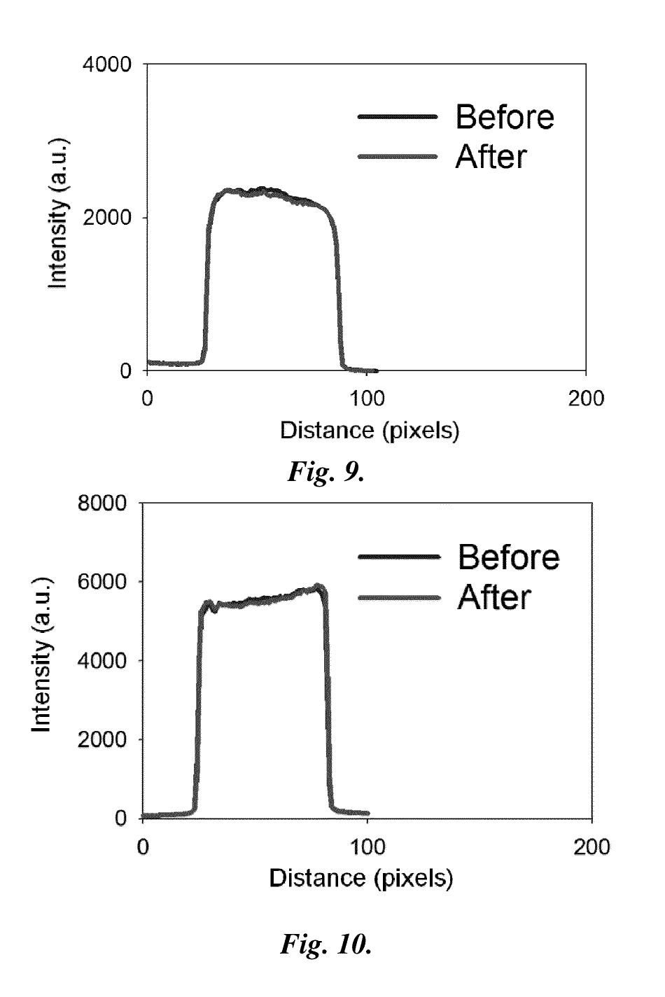

Sensing Avidin and Streptavidin Experiments were performed with POPC bilayers containing 5 mol % biotin PE and 0.5 mol % ortho-rhodamine B POPE in both increase and decrease mode. For these experiments, avidin and streptavidin were utilized as the binding analytes of interest. Both proteins have a strong affinity for biotin. Experiments were performed at pH 7.2, which falls within the linear response range of the dye as a function of pH (FIGS. 3A and 3B). Under these conditions, streptavidin carries a net negative charge while avidin carries a net positive charge. Experiments were run in three parallel microfluidic channels. Supported POPC bilayers containing ortho-rhodamine B-POPE and biotin PE were present in the first two channels. In the third channel, a control bilayer POPC bilayer was employed which contained ortho-rhodamine B-POPE, but no biotin PE (FIG. 4A). Fluorescent micrographs were captured every five minutes as 10 mM PBS buffer at pH 7.2 with 150 mM NaCl was flowed through the channels until fluorescence stabilization was achieved. Next, 300 nM of streptavidin was introduced into left channel, while 300 nM of avidin was flowed into the middle channel and right channels (30 min. mark). Data were collected every five minutes. The fluorescence intensity values from each channel as a function of time are shown in FIG. 4B. Moreover, the individual fluorescence line scans at 0 and 65 minute marks are shown in FIGS. 4C-4E. As can be seen, the introduction of streptavidin into the left channel caused the fluorescence to increase by about 5%. On the other hand, the addition of avidin into the middle channel resulted in a decrease in fluorescence intensity by about 20%. The control channel (on the right in the image) showed no change in signal upon the addition of avidin. An additional control experiment using streptavidin in the absence of biotin-PE ligands also showed no change in fluorescence (FIG. 9). The difference in fluorescence changes elicited by streptavidin and avidin reflects the isoelectric point of each protein at the operating pH of the assay. Indeed, the experiments were conducted just above the isoelectric point for streptavidin, but far below the isoelectric point for avidin. Consequently, avidin should be more highly charged under these conditions and therefore should perturb the apparent local pH above the supported lipid bilayer to a greater extent upon binding. Finally, it was necessary to ensure that the decrease sensor for avidin was indeed due to a decrease in the local hydronium ion concentration caused by protein binding rather than mere photobleaching of the dye. To do this, control experiments were carried out on biotinylated membranes utilizing the pH-insensitive para isomer of rhodamine B-labeled POPE with 5 mol % biotin-PE. As expected, the fluorescence intensity did not change in this assay (FIG. 10).

Altering Buffer Conditions to Optimize Assay

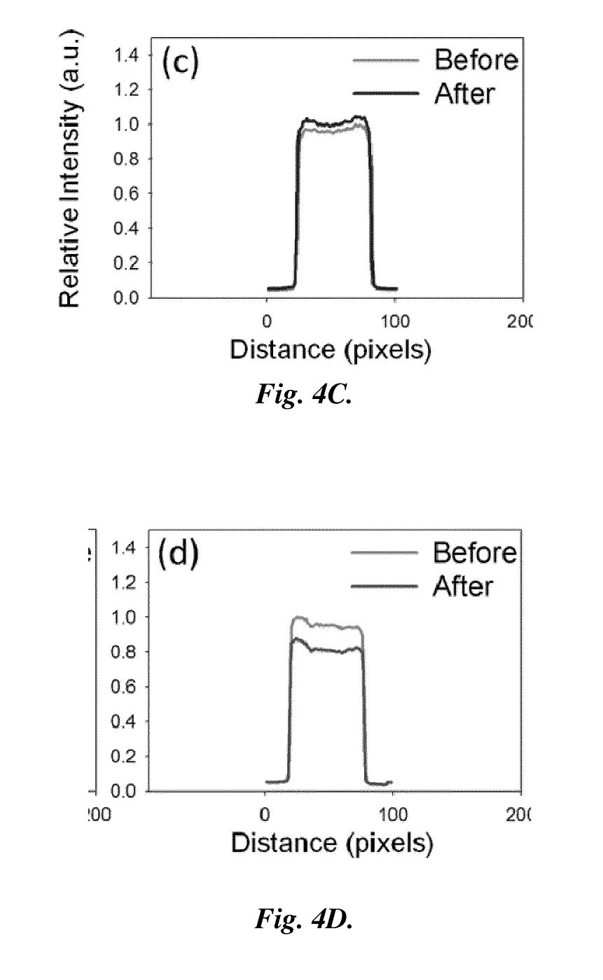

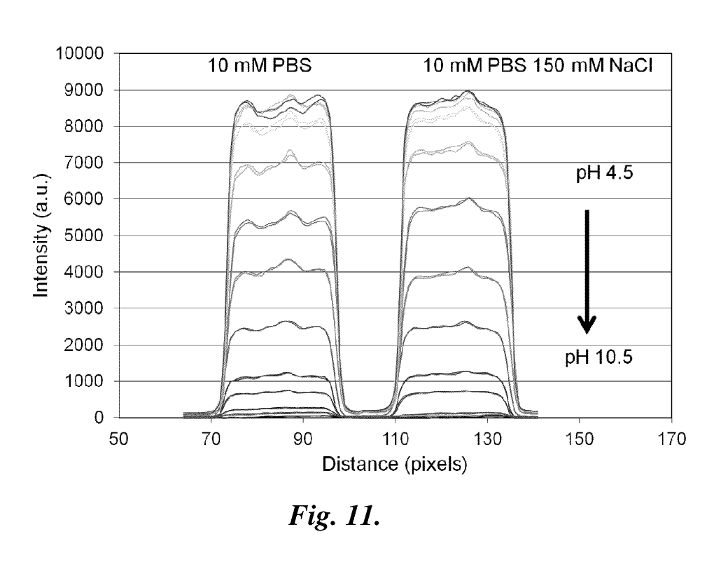

To probe the effect of salt screening on fluorescence response, experiments were carried out using 10 mM PBS without NaCl. This resulted in a buffer with decreased ionic strength and consequentially an increased Debye length. This should enhance the sensitivity of the assay by increasing the area of the bilayer that will be influenced by the binding of a protein. Specifically, under high salt conditions (150 mM NaCl) in a 10 mM PBS buffer, the Debye length should be about 6.6 Å. On the other hand, when the 10 mM PBS buffer is used without NaCl, the Debye length is about 12.4 Å. Thus decreasing the salt concentration allows each surface-bound protein to influence a greater range of dye molecules in its proximity. Titration curves performed under low and high salt concentrations were performed (FIG. 5). These experiments were run in the absence of protein molecules in bilayers consisting of 0.5 mol % ortho-rhodamine B and 5 mol % biotin-PE in POPC. As can be seen, the low salt concentration conditions resulted in a shift in the apparent pKa of the ortho-rhodamine B dye by about 1 pH unit (the line scans from which these titration curves were created can be seen in FIG. 11). This shift is not surprising considering that decreasing the ionic strength of a solution should increase the apparent pKa of a titratable species as predicted by Debye-Hückel theory. As a consequence of lowering the salt concentration, the linear pH responsive range of the assay is shifted to a higher value. In fact, this allows the assay to be run at pH 8.1, where streptavidin is more negatively charged, but avidin is somewhat less positively charged as described below.

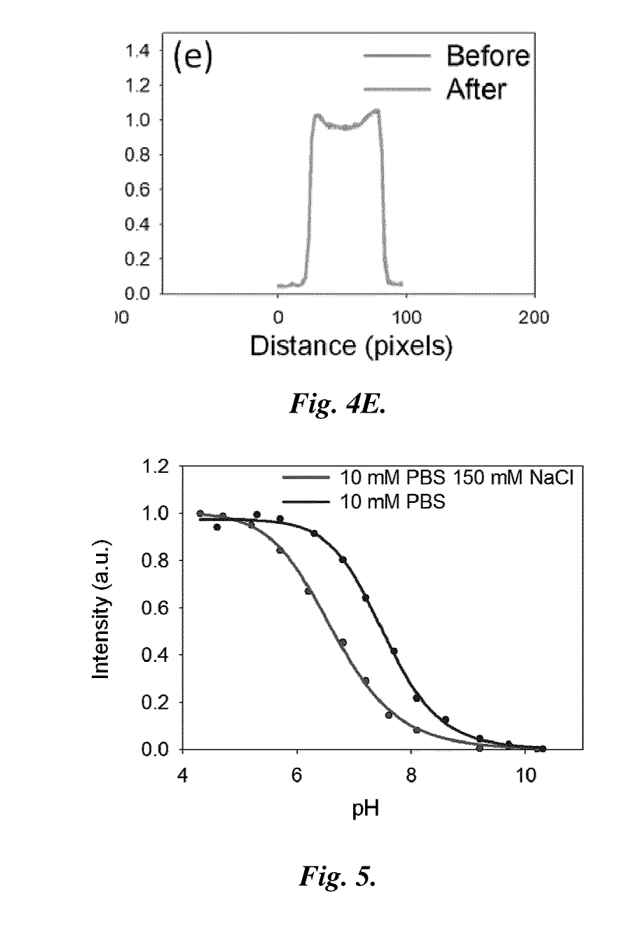

To test the effects of the low ionic strength conditions on sensor response, bilayers were formed inside two parallel microfluidic channels with and without 5 mol % biotin-PE. The bilayers were rinsed with 10 mM PBS until the fluorescence stabilized. Next, 300 nM streptavidin was introduced above the supported bilayers in a pH 8.1 buffer composed of 10 mM PBS and 150 mM NaCl and flowed for 30 minutes until the fluorescence response stabilized. Finally, the channels were flushed with 10 mM PBS buffer until the signal stabilized again. Identical experiments were also performed with avidin. As can be seen in FIG. 6, the introduction of streptavidin resulted in an increase in fluorescence whereas the introduction of avidin resulted in a fluorescence decrease just as in FIG. 4; however, the relative signal changes were greater (see Table 1).

TABLE 1
Protein signal changes for various experimental conditions.

| Protein | 10 mM PBS Buffer | Average % Change in Signal |
|---|---|---|
| (300 nM) | Conditions | upon Protein Binding |
| Streptavidin | pH 7.2 w/ 150 mM NaCl | +7 |
| | pH 8.1 | +21 |
| | pH 5.9 w/ 150 mM NaCl | −5 |
| Avidin | pH 7.2 w/ 150 mM NaCl | −12 |
| | pH 8.1 | −74 |

As can be seen from Table 1, the results from the low ionic strength experiments were remarkable. The percentage change in fluorescence upon the introduction of streptavidin was approximately a factor of three greater than when 150 mM NaCl was present. The change for avidin was even larger, a factor of six. Avidin should contain a smaller charge at pH 8.1 compared with 7.2, as it is closer to its isoelectric point. As such, the relatively large change in signal for avidin must be dominated by the increase in the Debye length under these conditions.

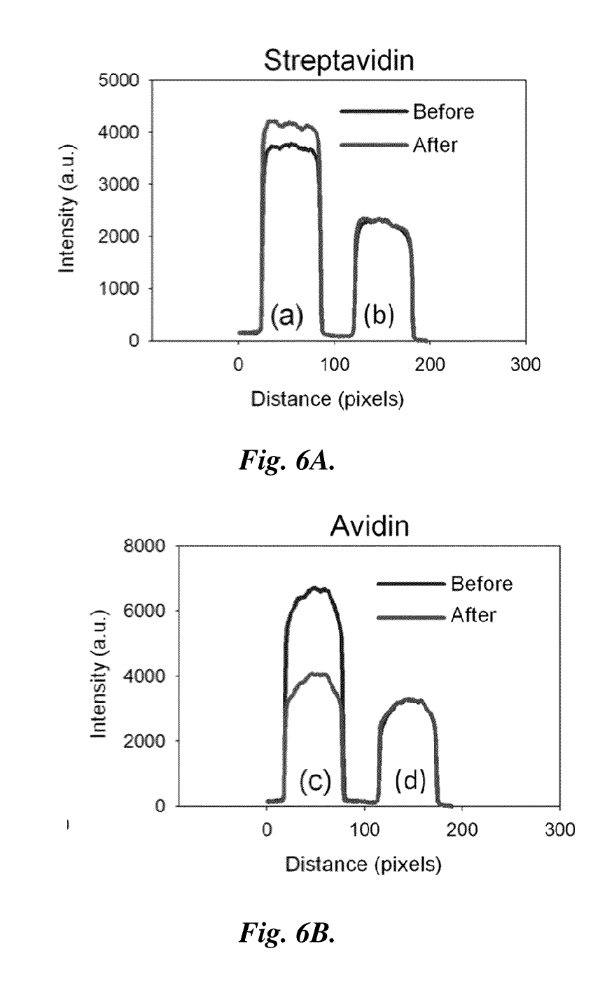

To explore the role of the assay's pH relative to the pI value of a protein analyte, a set of experiments was designed in which the fluorescence response was monitored as a function of pH at constant ionic strength for streptavidin. Because the isoelectric point for this protein is just below neutral pH, it is expected that the assay should switch from being an increase to decrease sensor when the experiments are run at 5.9 instead of 7.3. To test this idea, supported POPC bilayers were form with 0.5 mol % ortho-rhodamine B and 5 mol % biotin-PE in two parallel microfluidic channels. In each case, the solution contained 10 mM PBS with 150 mM NaCl. The normalized fluorescence intensity from these channels are provided in FIG. 7. For the first 20 minutes of each run, pure buffer at a given pH was flowed over the bilayer. This provided a fluorescence intensity benchmark with which to compare protein-bound conditions. Following stabilization, 300 nM streptavidin was introduced into the respective rinsing buffer and monitored. As can be seen, a fluorescence intensity increase was found at pH 7.3, but pH 5.9 showed a decrease. The latter result is consistent with the idea that the protein is below its pI of 6.4.

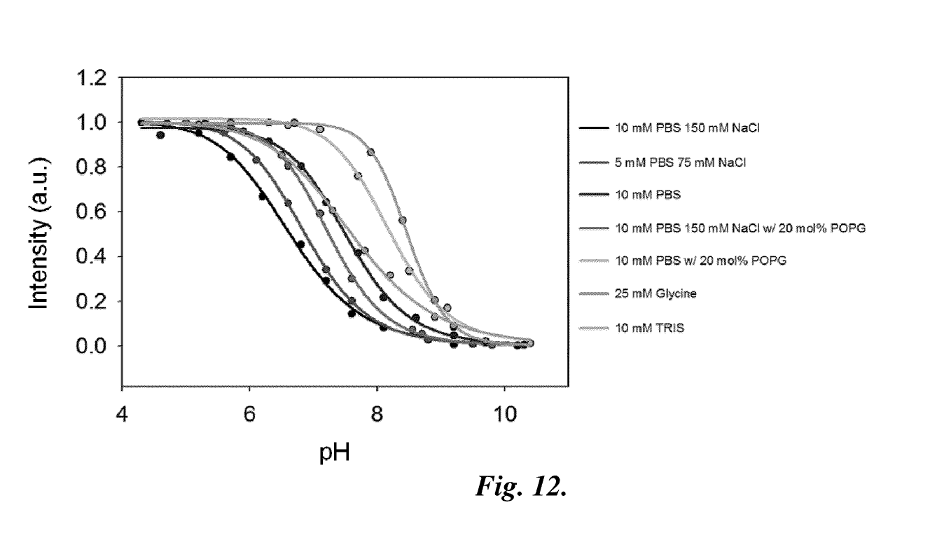

Two important considerations needed to be taken into account when employing the pH modulation sensor described herein. First, it is necessary to operate this assay in a pH window where the fluorescence response can be modulated. Second, it is important that proteins of interest be tested sufficiently far away from their pI values to elicit a fluorescence response. Through manipulation of the ionic strength of the operating buffer, it is possible to widen the assay range in order to accommodate proteins with a variety of different pI values. However, it should also be noted that the surface chemistry of the membrane and the underlying solid support play an important role in determining the surface potential. These variables too could be modulated by changing the substrate type or specific lipid membrane chemistry. FIG. 12 demonstrates that the addition of charged lipids to the membrane, the addition of osmolytes to the buffer, or even changing the nature of the buffer ions can all affect the titration curve for the dye. Of course, modifications to the dye itself would also change the range in which the assay can be operated.

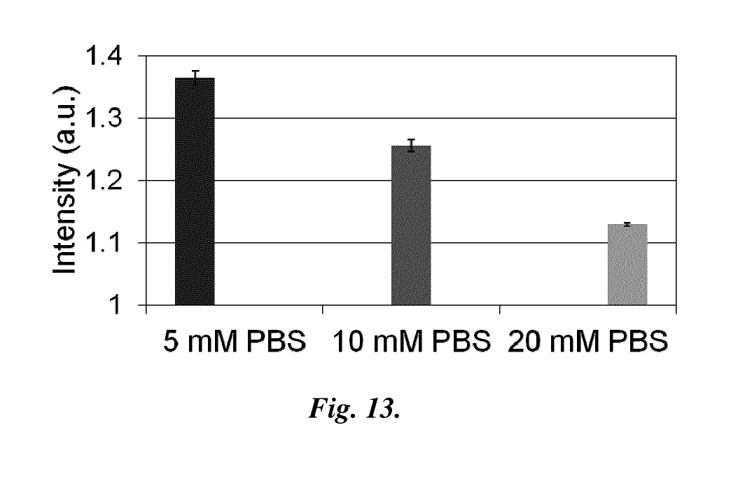

Although numerous variables can be manipulated to change the operating range of the assay, it should also be noted that changing these variables may also modulate the detection limits of this assay. Such behavior can also be quite complex. Indeed, in the case of avidin, running the assay at lower ionic strength, but higher pH (8.1 instead of 7.3), resulted in greater signal response despite the fact that the protein was actually closer to its isoelectric point (FIG. 6). In this particular case, lower ionic strength was the determining factor. Moreover, it could also be shown that decreasing the ionic strength of the buffer at constant pH increased the sensitivity for streptavidin detection (FIG. 13).

This pH modulation sensor represents a facile and rapid method for the detection of binding events on supported lipid bilayers and may serve as a preliminary screening assay monitoring protein-ligand interactions as well as small molecule or peptide interactions with bilayers. Additionally, the use of this technique in conjunction with other label-free techniques such as QCM-D may be able to offer quantitative insight into the nature of the pH-modulated fluorescent signal associated with binding events, as well as the effect that bilayer chemistries have on these responses.

Supported lipid membranes containing ortho-conjugated rhodamine B-POPE (1-hexadecanoyl-2-(9Z-octadecenoyl)-sn-glycero-3-phosphoethanolamine), which fluoresces in its protonated but not in its deprotonated form, were utilized as sensor platforms for biotin-avidin and biotin-streptavidin binding events. The membranes contained 5 mol % biotin-PE (1,2-dipalmitoyl-sn-glycero-3-phosphoethanolamine-N-(cap biotinyl) (sodium salt) as a capture ligand. The binding of avidin, which is positively charged at pH 7.2, made the surface charge more positive, which deprotonated the ortho-rhodamine B dye. This resulted in reduced fluorescence and consequently created a decrease sensor. The binding of streptavidin, which is negatively charged at pH 7.2, had the opposite effect. Reduction of the ionic strength by removing 150 mM NaCl from the 10 mM phosphate buffered saline (PBS) solution raised the apparent pKa of the ortho-rhodamine B by about 1 pH unit. This could be exploited in conjunction with bulk pH modulation to make the rhodamine B-POPE dye into either an increase or decrease fluorescent sensor for streptavidin, because its pI value is just below neutral pH.

In one embodiment, the present provides a method that utilizes the ortho isomer of rhodamine B (Lissamine rhodamine B sulfonyl chloride, mixed isomers) conjugated to the free amine of POPE (1-hexadecanoyl-2-(9Z-octadecenoyl)-sn-glycero-3-phosphoethanolamine) as a bilayer-conjugated reporter dye. The ortho rhodamine B isomer exhibits much more complete quenching behavior at high pH compared to ortho-TEXAS RED®. Additionally, the ortho isomer of rhodamine B exhibits pH-sensitive fluorescence in a more acidic pH range and is far less expensive than TEXAS RED®. Significantly, this more acidic dye can be exploited to detect positively charged as well as negatively proteins near neutral pH. For positively charged proteins, the sensor works in a manner similar to the detection of negatively charged species, except that a decrease in fluorescence accompanies a binding event because the interfacial potential becomes more positive and the effective local pH is therefore increased. Avidin and streptavidin proteins were employed to test the decrease and increase modalities, respectively, (FIG. 1) as these proteins demonstrate a high affinity for the same ligand (biotin) but have differing isoelectric points. Avidin has an isoelectric point (pI) of about 10. It is therefore positively charged at a working pH of 7.2 and, in fact, caused a decrease in the fluorescence response from the bilayer upon protein binding. For streptavidin, there was an increase in the fluorescence upon binding near or above the protein's reported isoelectric point of 6.4. On the other hand, a decrease in fluorescence accompanied streptavidin binding at pH values below the pI. Finally, by modulating the ionic strength of the buffer employed in this assay, it was possible to both increase sensitivity and shift the apparent pKa of the ortho-rhodamine B dye to more basic values.

The following examples are presented for the purpose of illustrating, not limiting, the invention.

### Examples

Materials.

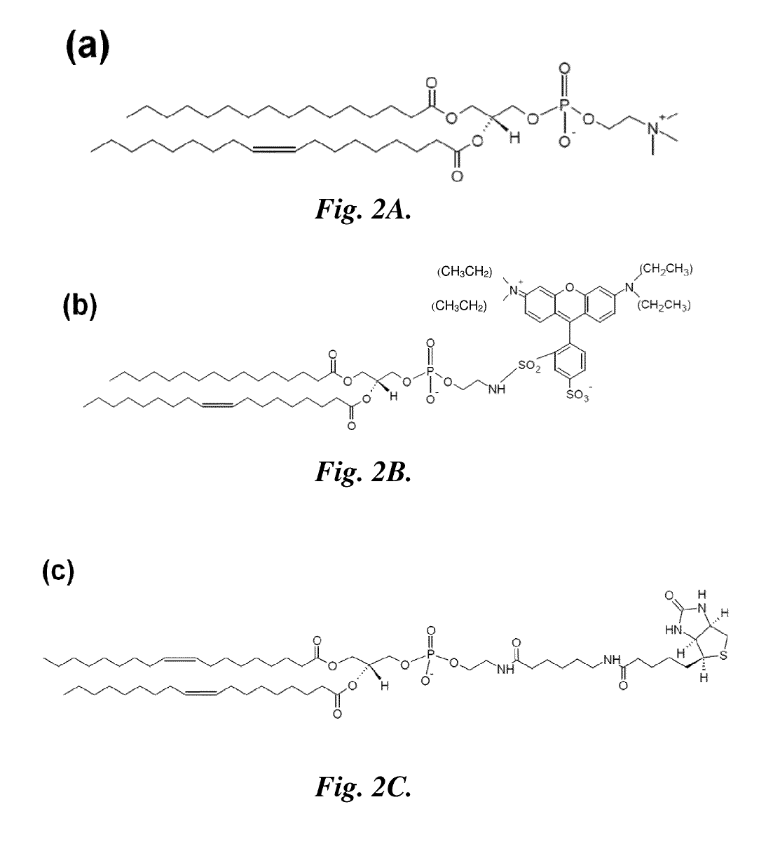

Lissamine rhodamine B sulfonyl chloride was obtained from Invitrogen (Eugene, Oreg.). POPC (1-palmitoyl-2-oleoyl-sn-glycero-3-phosphocholine), biotin-PE, and POPE were purchased from Avanti Polar Lipids (Alabaster, Ala.). The chemical structures of POPC, ortho-conjugated rhodamine B-POPE, and biotin-PE are shown in FIGS. 2A-2C, respectively. Purified water was obtained from a NANOpure Ultrapure Water System (18.2 MΩ·cm, Barnstead, Dubuque, Iowa). Glass coverslips (24×40 mm, No. 1.5, Corning Inc.) were used as planar solid supports. PDMS (polydimethylsiloxane, Dow Corning Sylgard Silicone Elastomer-184) was obtained from Krayden, Inc (El Paso, Tex.). Streptavidin and avidin were purchased from Rockland (Gilbertsville, Pa.).

Rhodamine B-POPE Conjugation and Separation.

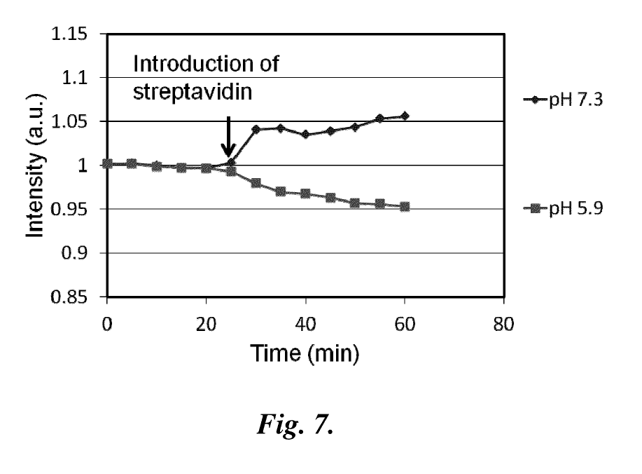

A solution of 10 mg of rhodamine B sulfonyl chloride in 1 mL of chloroform was mixed with 10 mg of POPE and 2 μL of diethylamine. The reaction was stirred for 1 h at room temperature. After the reaction, the solution was spotted onto a TLC plate (EMD, 5715-7, silica gel 60 F254) and developed in a 92:8 mixture by volume of chloroform to methanol to yield separated rhodamine-B labeled POPE isomers. The ortho-rhodamine B-POPE isomer was further purified on a second TLC plate developed with an ammonium hydroxide solution: dichloromethane: n-propanol mixture of 5:60:35 by volume. The successful labeling of the phospholipid was confirmed with matrix-assisted laser desorption ionization mass spectrometry, which can be seen in FIG. 8.

Small Unilamellar Vesicles (SUVs).

To make SUVs, lipids of the desired molar composition were mixed in chloroform and then dried by purging with nitrogen gas. Following desiccation under vacuum for at least 1 h, 0.5 mg/mL lipid suspensions were obtained by hydration with 10 mM phosphate-buffered saline (PBS) containing 150 mM NaCl. The SUV solutions were prepared by ten freeze-thawing cycles with liquid nitrogen and warm water followed by ten extrusion cycles through two stacked 0.1 μm polycarbonate membranes (Whatman) using a Lipex extruder (Northern Lipids Inc., Vancouver, Canada). The size of the SUVs was determined to be 90±10 nm in diameter by dynamic light scattering (Brookhaven Instruments 90Plus Particle Size Analyzer).

Microfluidic Device Fabrication.

Microfluidic devices consisting of a PDMS (polydimethylsiloxane) mold created with a channel-patterned glass substrate were formed by soft lithographic methods. First, photoresist was spin coated onto clean glass slides (soda-lime glass slides, Corning) which were then exposed to UV light through a Kodak technical pan film photomask consisting of the appropriate microfluidic channel pattern. These photoresist-coated glass slides were treated in developing solution and baked overnight at 120° C. The thickness of the photoresist layer was determined to be 2.3 μm to 2.4 μm using a Dektek 3 Stylus Profilometer. The patterned glass slides were then etched by immersing them into a buffered oxide etchant (BOE) solution. The BOE solution was prepared by mixing 48% HF (EMD Chemicals Inc., Germany) and aqueous NH₄F (200 g/300 mL purified water, Alfa Aesar, Ward Hill, Mass.) in a 1:6 volume ratio. The etching process was repeated until the depth of the channels was between 15 μm and 19 μm. The slides were then washed with acetone to remove the remaining photoresist and dried under flowing nitrogen gas. Next, PDMS (Sylgard silicone elastomer-184) and its cross-linking agent (10:1 volume ratio) were mixed in a plastic beaker and degassed inside a desiccator under vacuum. The liquid PDMS mixture was poured over a patterned glass slide and allowed to cure in an oven at 70° C. After 1 h, the nascently-formed PDMS elastomer was peeled off of the glass slide. This mold and a freshly cleaned glass coverslip were placed into a 25 W oxygen plasma cleaner (PDC-32G, Harrick) for 30 s and were then immediately brought into contact to form the PDMS/glass microfluidic device. It should be noted that both the glass slides and coverslips used in these procedures were cleaned in a boiling solution of ICN 7X detergent (Costa Mesa, Calif.) mixed with purified water at a 1:4 volume ratio. Following this, the slides were annealed in a kiln (Sentry Xpress 2.0, Orton Ceramic Foundation, OH) at 500° C. for 5 h before the oxygen plasma treatment.

Supported Lipid Bilayers (SLBs).

SLBs were formed within the channels of microfluidic devices via spontaneous vesicle fusion. Five μL of an SUV solution was injected into each microchannel and incubated for 10 min followed by rinsing with pure PBS buffer to remove excess vesicles. For protein detection measurements, solutions of either avidin or streptavidin were continually flowed through each channel for up to 1 h.

pH Titration of SLBs.

PBS solutions with and without 150 mM NaCl were prepared at pH values ranging from 4.5 to 10.5 by mixing with appropriate amounts of Na₂HPO₄, NaH₂PO₄, or Na₃PO₄. The pH was measured with a standard glass electrode setup having an absolute error of ±0.1 pH units. Titration curves of the fluorescence from the dye molecules in the SLBs were obtained by systematically changing the pH of the bulk solution by flowing the appropriate pH adjusted buffer solutions through the microfluidic device in a stepwise fashion. The value of the pH for each buffer was determined immediately prior to adding it to the microfluidic channels.

Fluorescence Microscopy.

All fluorescence images were obtained using a Nikon Eclipse Ti-U fluorescence microscope (Tokyo, Japan) equipped with a ProEM 1024 CCD camera (Princeton Instruments). A 10× air objective (N.A.=0.45) was used for all detection measurements. The light source was a Lumen 200 (Prior Scientific) and a TEXAS RED® filter set (Chroma Technology, Bellows Falls, Vt.) was employed. All images were processed with MetaMorph software (Version 7.7.0.0, Universal Imaging).

While illustrative embodiments have been illustrated and described, it will be appreciated that various changes can be made therein without departing from the spirit and scope of the invention.

The embodiments of the invention in which an exclusive property or privilege is claimed are defined as follows:

1. A method for sensing the binding of an analyte to a surface, comprising:
(a) contacting an analyte with a surface, wherein the analyte is a second binding partner of a binding pair, wherein the surface comprises a first binding partner of the binding pair having a binding affinity toward the second binding partner, and wherein the surface further comprises a pH-sensitive fluorescent agent having a first fluorescence emission intensity at a first pH and a second fluorescence emission intensity at a second pH; and
(b) measuring the fluorescence emission intensity of the pH-sensitive fluorescent agent after contacting the surface with the analyte, wherein a change in the fluorescent emission intensity after contacting the surface with the analyte indicates binding of the analyte to the surface.

2. The method of claim 1, wherein the surface is a lipid bilayer.

3. The method of claim 1, wherein the surface is a lipid bilayer of a liposome and the sensing of the binding of the analyte to the surface occurs in bulk solution.

4. The method of claim 1, wherein the surface is a supported lipid bilayer.

5. The method of claim 1, wherein the first binding partner is covalently coupled to the lipid bilayer.

6. The method of claim 1, wherein the first binding partner is covalently coupled to a component making up the lipid bilayer.

7. The method of claim 1, wherein the first binding partner is a ligand and the second binding partner is a receptor.

8. The method of claim 1, wherein the first binding partner is a receptor and the second binding partner is a ligand.

9. The method of claim 1, wherein the first binding partner is an antigen and the second binding partner is an antibody or fragment thereof.

10. The method of claim 1, wherein the first binding partner is an antibody or fragment thereof and the second binding partner is an antigen.

11. The method of claim 1, wherein the first binding partner is a nucleic acid and the second binding partner is a nucleic acid.

12. The method of claim 1, wherein the pH-sensitive fluorescent agent operative in the method from a pH of about 5.5 to about 9.0.

13. The method of claim 1, wherein the pH-sensitive fluorescent agent is covalently coupled to the lipid bilayer.

14. The method of claim 1, wherein the pH-sensitive fluorescent agent is covalently coupled to a component making up the lipid bilayer.

15. The method of claim 1, wherein the first fluorescence emission intensity is greater than the second fluorescence emission intensity.

16. The method of claim 1, wherein the first fluorescence emission intensity is less than the second fluorescence emission intensity.

17. The method of claim 1, wherein pH-sensitive fluorescent agent is a rhodamine, selected from the group consisting of an ortho-rhodamine B, and mixtures thereof.

18. The method of claim 1, wherein the surface is the surface of a sensor.

19. A surface, comprising:
(a) one or more first binding partners, each first binding partner having a binding affinity to a second binding partner of a binding pair; and
(b) one or more pH-sensitive fluorescent agents, wherein each pH-sensitive fluorescent agent has a first fluorescent emission intensity at a first pH and a second fluorescent emission intensity at a second pH, wherein the first and second emission intensities are not the same.

20. A method for detecting the presence of an analyte in a sample, comprising:
(a) contacting a sample with a surface, wherein the surface comprises a first binding partner of a binding pair having a binding affinity toward a second binding partner, wherein the analyte is the second binding partner, and wherein the surface further comprises a pH-sensitive fluorescent agent having a first fluorescence emission intensity at a first pH and a second fluorescence emission intensity at a second pH; and
(b) measuring the fluorescence emission intensity of the pH-sensitive fluorescent agent after contacting the surface with the sample, wherein a change in the fluorescent emission intensity after contacting the surface with the sample indicates binding of the analyte to the surface and the presence of the analyte in the sample.
